(19) United States
(12) Patent Application Publication
(10) Pub. No.: US 2006/0211099 A1
(43) Pub. Date: Sep. 21, 2006

(54) L-carnitin dehydrogenases, their derivatives and method for producing substituted (s) alkanols

(75) Inventors: Henning Althöfer, Wachenheim (DE); Maria Kesseler, Mannheim (DE)

Correspondence Address: CONNOLLY BOVE LODGE & HUTZ, LLP, P O BOX 2207, WILMINGTON, DE, 19899, US

(73) Assignee: BASF AKTIENGESELLSCHAFT, Ludwigshafen (DE)

(21) Appl. No.: 10/552,218

(22) PCT Filed: Apr. 6, 2004
(86) PCT No.: PCT/EP04/03655

§ 371 Date: Oct. 6, 2005

(30) Foreign Application Priority Data
Apr. 7, 2003 (DE) 103 15 760.3

(51) Int. Cl. C12P 17/00; C07H 21/04; C07D 333/22; C12N 1/21; C12N 1/18

(52) U.S. Cl. 435/117; 435/252.3; 435/254.1; 435/190; 536/23.2; 549/76

(57) ABSTRACT

The present invention relates to proteins having an enzymatic activity of reducing substituted alkanones such as 3-methylamino-1-(2-thienyl)-propan-1-one. The invention furthermore relates to nucleic acids coding for said proteins, nucleic acid constructs, vectors, genetically modified microorganisms and to methods for preparing substituted (S)-alkanols, such as, for example, (S)-3-methylamino-1-(2-thienyl)-(S)-propanol.

The present invention relates to proteins having an enzymatic activity for reducing substituted alkanones such as 3-methylamino-1-(2-thienyl)-propan-1-one. The invention furthermore relates to nucleic acids coding for said proteins, nucleic acid constructs, vectors, genetically modified microorganisms and to methods for preparing substituted (S)-alkanols, such as, for example, (S)-3-methylamino-1-(2-thienyl)-(S)-propanol.

### PRIOR ART

Dehydrogenases are versatile catalysts for the enantioselective reduction of aldehydes or ketones to give the corresponding alcohols. A distinction is made between (R)- and (S)-specific dehydrogenases. These catalysts are increasingly being used for industrial synthesis of optically active alcohols. Optical activity is the precondition of selective action of many pharmaceutical and agrochemical active compounds. Here, one enantiomer may have the desired action and the other enantiomer a genotoxic action. For this reason, synthesis of pharmaceutical and agrochemical active compounds employs catalysts having the required stereospecificity for preparing optically active alcohols.

3-Methylamino-1-(2-thienyl)-(S)-propanol (“Duloxetine alcohol”) is a building block in Duloxetine synthesis. Duloxetine is a pharmaceutical active compound which is currently going through the approval process and is intended to be used in the fields of indication of depression and incontinence.

Synthesis routes to Duloxetine alcohol and Duloxetine are described in the literature (cf. EP-A-0 273 658). These synthesis routes have the disadvantage that the synthesis results in a racemic alcohol mixture, requiring subsequent resolution of the racemate byating the racemconverte into a mixture of diastereomers via formation of a salt with an optically active counterion. The diastereomers are then physically separated. This results in high process costs, due to repeated separation of solids and liquids, and increased use of starting compounds, due to addition of an optically active salt for separation.

Stereospecific reduction of 3-methylamino-1-(2-thienyl)-propanone would provide a less expensive path to Duloxetine alcohol.

### BRIEF DESCRIPTION OF THE INVENTION

It is an object of the present invention to find a route to stereospecific reduction of substituted alkanones such as 3-methylamino-1-(2-thienyl)-propan-2-one.

We have found that this object is achieved by the surprising finding that enzymes having L-carnitine dehydrogenase activity are capable of catalyzing the above reaction in a stereospecific manner.

Firstly, the invention relates to a method for microbiological, in particular enantioselective, preparation of substituted (S)-alkanols of the formula I 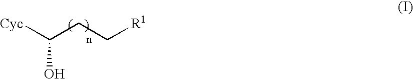
in which
n is an integer from 0 to 5, in particular 0, 1 or 2;
Cyc is an unsubstituted or substituted, mono- or polynuclear, saturated or unsaturated, carbocyclic or heterocyclic ring, in particular an unsubstituted or substituted, unsaturated, mononuclear heterocyclic ring, and
R¹ is halogen, SH, OH, NO₂, NR²R³ or NR²R³R⁴⁺X⁻, in particular halogen or NR²R³, where R², R³ and R⁴ independently of one another are H or a lower alkyl or lower alkoxy radical and X⁻ is a counterion,
   wherein, in a medium containing an alkanone of the formula II 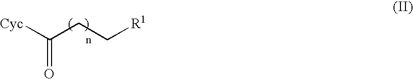
in which n, Cyc and R¹ are as defined above,
a) a microorganism producing an enzyme having L-carnitine dehydrogenase activity is cultured, or
b) an enzyme having L-carnitine dehydrogenase activity is incubated,
   the compound of the formula II being enzymatically reduced to give the compound of the formula I, and the essentially enantiomerically pure product formed is isolated.

In a particularly preferred embodiment, the method serves to prepare 3-methylamino-1-(2-thienyl)-(S)-propanol of the formula III 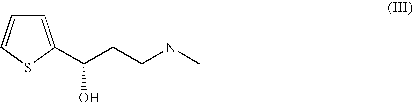
wherein, in a medium containing 3-methylamino-1-(2-thienyl)-propan-2-one of the formula IV 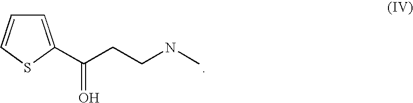
said compound is enzymatically reduced to give a compound of the formula III and the essentially enantiomerically pure product formed is isolated.

Preference is given to using in these methods an enzyme having L-carnitine dehydrogenase activity, which is selected from among L-carnitine dehydrogenases (E.C. 1.1.1.108) and 3-hydroxyacyl-CoA dehydrogenases (E.C. 1.1.1.35).

Enzymes of this kind having L-carnitine dehydrogenase activity are selected in particular from among enzymes of microorganisms of the genera Alcaligenes, Pseudomonas, Xanthomonas, Staphylococcus, Rhizobium, Agrobacterium, Streptomyces and Archaeglobus.

In a particularly preferred embodiment of the invention, the enzyme having L-carnitine dehydrogenase activity is selected from among enzymes comprising an amino acid sequence according to SEQ ID NO: 2, 3, 4, 5, 6, 7, 8, 9 or 10 or encoded by nucleic acid sequences derived therefrom; and functional equivalents of said enzymes, which have L-carnitine dehydrogenase activity and catalyze the enantioselective synthesis of a compound of the formula I.

For example, the enzyme having L-carnitine dehydrogenase activity may be encoded by a nucleic acid sequence according to SEQ ID NO:1 or a functional equivalent thereof.

Preference is given to carrying out the method of the invention with addition of reduction equivalents (NADH or NADPH) or under (biochemical or electrochemical) conditions under which the reduction equivalents consumed in the reaction are regenerated.

Furthermore, preference is given to allowing the compound of the formula II, for example of the formula IV, to be reacted in the presence of a microorganism selected from among bacteria of the families Enterobacteriaceae, Pseudomonadaceae, Rhizobiaceae, Streptomycetaceae and Nocardiaceae. Said microorganism may in particular be a recombinant microorganism which has been transformed with a nucleic acid construct coding for an enzyme having L-carnitine dehydrogenase activity as defined above.

In particular, the invention relates to a method as defined above, wherein
a) a microorganism producing an enzyme having L-carnitine dehydrogenase activity is isolated from a natural source or is prepared recombinantly,
b) said microorganism is propagated,
c) said enzyme having L-carnitine dehydrogenase activity is, where appropriate, isolated from said microorganism or a protein fraction containing said enzyme is prepared from said microorganism, and
d) said microorganism according to stage b) or said enzyme according to stage c) is transferred into a medium containing a compound of the formula I.

The invention furthermore relates to a compound of the formula V 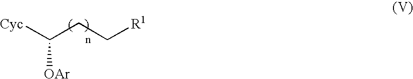
in which n, Cyc and R¹ are as defined above and Ar is a mono- or polynuclear, unsubstituted or substituted aryl radical, and
wherein
a) first a compound of the formula I is prepared microbiologically as defined in any of the preceding claims; and
b) the compound of the formula I is reacted with an aromatic compound of the formula VI
   Ar—Y (VI)
   in which Ar is as defined above and Y is a leaving group, and
c) the compound of the formula V is isolated and, where appropriate, converted to a pharmaceutically acceptable acid addition salt such as oxalates, for example.

Preference is given here to preparing a compound of the formula V in which Ar is 1-naphthyl, Cyc is 2-thienyl, R¹ is monomethylamino and n is 1.

The invention further relates to polypeptides which comprise an amino acid sequence according to SEQ ID NO: 2, 3, 4, 5, 6, 7, 8, 9 or 10 or are encoded by nucleic acid sequences derived therefrom; and to functional equivalents of these enzymes, which have L-carnitine dehydrogenase activity and which catalyze the enantioselective synthesis of a compound of the formula I and/or III.

The invention moreover relates to coding nucleic acid sequences comprising the sequence coding for a polypeptide as defined above.

The invention furthermore relates to expression cassettes comprising a coding nucleic acid sequence as defined above and operatively linked to at least one regulatory nucleic acid sequence.

The invention further relates to recombinant vectors comprising at least one such expression cassette.

The invention also relates to prokaryotic or eukaryotic hosts transformed with at least one vector of the invention.

Finally, the invention relates to the use of an enzyme having L-carnitine dehydrogenase activity as defined above or of a microorganism producing said enzyme for preparing compounds of the formula I or III, in particular for preparing Duloxetine of the formula VII

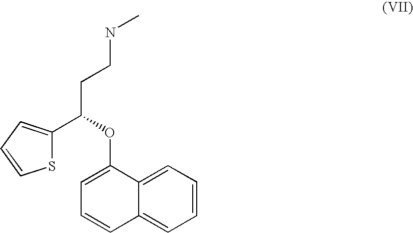

### DETAILED DESCRIPTION OF THE INVENTION

A. General Terms and Definitions

Unless specified otherwise, the following general meanings apply:

“Halogen” is fluorine, chlorine, bromine or iodine, in particular fluorine or chlorine.

“Lower alkyl” is straight-chain or branched alkyl radicals having 1 to 6 carbon atoms, such as methyl, ethyl, isopropyl or n-propyl, n-butyl, isobutyl, sec- or tert-butyl, n-pentyl or 2-methylbutyl, n-hexyl, 2-methylpentyl, 3-methylpentyl, 2-ethylbutyl.

“Lower alkenyl” is the mono- or polyunsaturated, preferred mono- or diunsaturated, analogs of the abovementioned alkyl radicals having 2 to 6 carbon atoms, it being possible for the double bond to be in any position of the carbon chain.

“Lower alkoxy” is the oxygen-terminated analogs of the above alkyl radicals.

“Aryl” is a mono- or polynuclear, preferably mono- or binuclear, unsubstituted or substituted aromatic radical, in particular phenyl or a naphthyl bound via any ring position, such as 1- or 2-naphthyl. Where appropriate, these aryl radicals may carry 1 or 2 identical or different substituents selected from among halogen, lower alkyl, lower alkoxy as defined above or trifluoromethyl.

B. Substituted Alkanones, (S)-alkanols and Derivatives Thereof

Alkanols accessible by enzymatic catalysis according to the invention are those of the above formula (I) in which
n is an integer from 0 to 5;
Cyc is an unsubstituted or substituted, mono- or polynuclear, saturated or unsaturated, carbocyclic or heterocyclic ring, and
R¹ is halogen, SH, OH, NO₂, NR²R³ or NR²R³R⁴⁺X⁻, where R², R³ and R⁴ independently of one another are H or a lower alkyl or lower alkoxy radical and X⁻ is a counterion.

The alkanols of the above formula II, used for enzymatic synthesis, are compounds known per se and obtainable with application of well-known organic synthesis methods (cf. e.g. EP-A- 0 273 658).

In the above compounds, n is preferably 0, 1 or 2, in particular 1.

Examples of carbo- and heterocyclic groups Cyc which should be mentioned are in particular mono- or binuclear, preferably mononuclear, groups having up to 4, preferably 1 or 2, identical or different ring heteroatoms selected from among O, N and S:

Said carbo- or heterocyclic rings comprise in particular from 3 to 12, preferably 4, 5 or 6, ring carbon atoms. Examples which may be mentioned are cyclopropyl, cyclobutyl, cyclopentyl, cyclohexyl, cycloheptyl, the mono- or polyunsaturated analogs thereof such as cyclobutenyl, cyclopentenyl, cyclohexenyl, cycloheptenyl, cyclohexadienyl, cycloheptadienyl; and also 5- to 7-membered saturated or mono- or polyunsaturated heterocyclic radicals having from 1 to 4 heteroatoms selected from among O, N and S, it being possible for the heterocycle to be fused to another heterocycle or carbocycle, where appropriate. Radicals which should be mentioned here are in particular radicals derived from pyrrolidine, tetrahydrofuran, piperidine, morpholine, pyrrole, furan, thiophene, pyrazole, imidazole, oxazole, thiazole, pyridine, pyran, pyrimidine, pyridazine, pyrazine, coumarone, indole and quinoline.

The radicals Cyc may be bound here via any ring position, preferably via a ring carbon atom, to the alkanone or the alkanol.

Examples of suitable Cyc radicals are 2-thienyl, 3-thienyl; 2-furanyl, 3-furanyl; 2-pyridyl, 3-pyridyl or 4-pyridyl; 2-thiazolyl, 4-thiazolyl or 5-thiazolyl; 4-methyl-2-thienyl, 3-ethyl-2-thienyl, 2-methyl-3-thienyl, 4-propyl-3-thienyl, 5-n-butyl-2-thienyl, 4-methyl-3-thienyl, 3-methyl-2-thienyl; 3-chloro-2-thienyl, 4-bromo-3-thienyl, 2-iodo-3-thienyl, 5-iodo-3-thienyl, 4-fluoro-2-thienyl, 2-bromo-3-thienyl and 4-chloro-2-thienyl.

The radicals Cyc may furthermore be mono- or polysubstituted, for example mono- or disubstituted. The substituents are preferably located on a ring carbon atom. Examples of suitable substituents are halogen, lower alkyl, lower alkenyl, lower alkoxy, —OH, —SH, —NO₂ or NR²R³, where R² and R³ are as defined above, preferably halogen or lower alkyl.

R¹ is in particular halogen, NR²R³ or NR²R³R⁴⁺X⁻, where R², R³ or R², R³ and R⁴ are independently of one another H or a lower alkyl or lower alkoxy radical and X⁻ is a counterion, any of the radicals R², R³ and R⁴ being preferably H. Examples of suitable counterions are acid anions as obtained, for example, from preparation of an acid addition salt. Examples thereof are mentioned, for example, in EP-A-0 273 658 which is hereby incorporated by reference. Preferred examples of radicals R¹ are in particular fluorine or chlorine and also NR²R³ in which R² and R³ are identical or different and are H or methyl, ethyl or n-propyl; particularly preferably, R¹ is chlorine or —NHmethyl.

C. Enzymes having L-carnitine Dehydrogenase Activity

The inventive enzyme having L-carnitine dehydrogenase activity is in particular selected from L-carnitine dehydrogenases (E.C. 1.1.1.108) and 3-hydroxyacyl-CoA dehydrogenases (E.C. 1.1.1.35) (cf. also Kleber H P (1997) FEMS Microbiology Letters 147 1-9).

Said L-carnitine dehydrogenases/hydroxyacyl-CoA dehydrogenases can be found in organisms, in particular microorganisms such as bacteria, yeasts or fungi. The enzyme or enzymes have a high enzymatic activity for reducing alkanones of the formula II, such as 3-methylamino-1-(2-thienyl)-propan-1-one to 3-methylamino-1-(2-thienyl)-(S)-propanol. The dehydrogenase likewise converts other substrates such as, for example, the dimethyl derivatives of the ketone as well as the monomethyl compounds.

Preferably, but without being limited thereto, enzymes of this kind can be obtained from microorganisms of the genera Alcaligenes, Pseudomonas, Xanthomonas, Staphylococcus, Rhizobium, Agrobacterium, Streptomyces and Archaeglobus.

Preferred enzymes having L-carnitine dehydrogenase activity comprise an amino acid sequence according to SEQ ID NO: 2, 3, 4, 5, 6, 7, 8, 9 or 10.

The invention likewise comprises “functional equivalents” of the specifically disclosed enzymes having L-carnitine dehydrogenase activity and the use of these in the methods of the invention.

“Functional equivalents” or analogs of the specifically disclosed enzymes are, for the purposes of the present invention, polypeptides which differ from said enzymes but still retain the desired biological activity such as substrate specificity, for example. Thus, for example, “functional equivalents” mean enzymes which reduce 3-methylamino-1-(2-thienyl)-propan-1-one to the corresponding S-alcohol and which have at least 20%, preferably 50%, particularly preferably 75%, very particularly preferably 90%, of the activity of an enzyme comprising any of the amino acid sequences listed under SEQ ID NO:2 to 10. Moreover, functional equivalents are preferably stable between pH 4 and 10 and advantageously have a pH optimum of between pH 5 and 8 and a temperature optimum in the range from 20° C. to 80° C.

“Functional equivalents” mean according to the invention in particular also mutants which have in at least one sequence position of the abovementioned amino acid sequences an amino acid different from the specifically mentioned amino acids but which have nevertheless one of the abovementioned biological activities. “Functional equivalents” thus comprise the mutants obtainable by one or more amino acid additions, substitutions, deletions and/or inversions, it being possible for said modifications to occur in any sequence position as long as they result in a mutant having the property profile of the invention. In particular, functional equivalence also exists when the reactivity patterns between mutant and unmodified polypeptide correspond qualitatively, i.e. when identical substrates are converted at different rates, for example.

“Functional equivalents” in the above sense are also “precursors” of the polypeptides described and also “functional derivatives” and “salts” of said polypeptides.

In this context, “precursors” are natural or synthetic precursors of the polypeptides with or without the desired biological activity.

The term “salts” means not only salts of carboxyl groups, but also acid addition salts of amino groups of the protein molecules of the invention. Salts of carboxyl groups can be prepared in a manner known per se and comprise inorganic salts such as, for example, sodium, calcium, ammonium, iron and zinc salts, and also salts with organic bases such as, for example, amines, such as triethanolamine, arginine, lysine, piperidine, and the like. The invention likewise relates to acid addition salts such as, for example, salts with mineral acids such as hydrochloric acid or sulfuric acid and salts with organic acids such as acetic acid and oxalic acid.

“Functional derivatives” of polypeptides of the invention may likewise be prepared on functional amino acid side groups or the N- or C-terminal end thereof with the aid of known techniques. Derivatives of this kind comprise, for example, aliphatic esters of carboxylic acid groups, amides of carboxylic acid groups, obtainable by reaction with ammonia or with a primary or secondary amine; N-acyl derivative of free amino groups, prepared by reaction with acyl groups; or O-acyl derivatives of free hydroxyl groups, prepared by reaction with acyl groups.

“Functional equivalents” also comprise, of course, polypeptides obtainable from other organisms and also naturally occurring variants. For example, regions of homologous sequences can be determined by sequence comparison, and equivalent enzymes can be established on the basis of the specific requirements of the invention.

“Functional equivalents” likewise comprise fragments, preferably individual domains or sequence motifs, of the polypeptides of the invention, which have the desired biological function, for example.

Moreover, “functional equivalents” are fusion proteins which have any of the abovementioned polypeptide sequences or functional equivalents derived therefrom and at least one other heterologous sequence functionally different therefrom in functional N- or C-terminal linkage (i.e. without substantial mutual functional impairment of the fusion protein moieties). Nonlimiting examples of heterologous sequences of this kind are signal peptides or enzymes, for example.

The invention also relates to “functional equivalents” which are homologs of the specifically disclosed proteins. These have at least 60%, preferably at least 75%, in particular at least 85%, such as, for example, 90%, 95% or 99%, homology to any of the specifically disclosed amino acid sequences, calculated by the algorithm of Pearson and Lipman, Proc. Natl. Acad, Sci. (USA) 85(8), 1988, 2444-2448. Percentage homology of a homologous polypeptide of the invention means in particular percentage identity of the amino acid residues based on the total length of one of the amino acid sequences specifically described herein.

In the event of a possible protein glycosylation, “functional equivalents” of the invention comprise proteins of the above-specified type in deglycosylated or glycosylated form and also modified forms obtainable by altering the glycosylation pattern.

Homologs of the proteins or polypeptides of the invention may be generated by mutagenesis, for example by point mutation or truncation of the protein.

Homologs of the proteins of the invention may be identified by screening combinatorial libraries of mutants such as truncation mutants, for example. For example, it is possible to generate a variegated library of protein variants by combinatorial mutagenesis at the nucleic acid level, for example by enzymatically ligating a mixture of synthetic oligonucleotides. There is a multiplicity of methods which can be used to prepare libraries of potential homologs from a degenerate oligonucleotide sequence. Chemical synthesis of a degenerate gene sequence may be carried out in a DNA synthesizer, and the synthetic gene can then be ligated into a suitable expression vector. The use of a degenerate set of genes makes it possible to provide, in one mixture, all sequences which encode the desired set of potential protein sequences. Methods for synthesizing degenerate oligonucleotides are known to the skilled worker (e.g. Narang, S. A. (1983) Tetrahedron 39:3; Itakura et al. (1984) Annu. Rev. Biochem. 53:323; Itakura et al., (1984) Science 198:1056; Ike et al. (1983) Nucleic Acids Res. 11:477).

The prior art discloses a plurality of techniques for screening gene products of combinatorial libraries which have been prepared by point mutations or truncation and for screening cDNA libraries for gene products with a selected property. These techniques can be adapted to the rapid screening of the gene libraries which have been generated by combinatorial mutagenesis of homologs of the invention. The most frequently used techniques for screening large gene libraries subjected to high-throughput analysis comprise cloning of the gene library into replicable expression vectors, transforming suitable cells with the resulting vector library and expressing the combinatorial genes under conditions under which detection of the desired activity facilitates isolation of the vector encoding the gene whose product has been detected. Recursive ensemble mutagenesis (REM), a technique which increases the frequency of functional mutants in the libraries, may be used in combination with the screening assays in order to identify homologs (Arkin and Yourvan (1992) PNAS 89:7811-7815; Delgrave et al. (1993) Protein Engineering 6(3):327-331).

D. Coding Nucleic Acid Sequences

In the context of the present invention, the terms “to express” or “overexpression” describe production of or increase in intracellular activity of one or more enzymes in a microorganism, which are encoded by the corresponding DNA. For this purpose, for example, it is possible to introduce a gene into an organism, to replace an existing gene with a different gene, to increase the copy number of the gene or genes, to use a strong promoter or to use a gene coding for a corresponding enzyme having a high activity, and it is possible to combine these measures, where appropriate.

The invention relates in particular to nucleic acid sequences coding for an enzyme having L-carnitine dehydrogenase activity. Preference is given to nucleic acid sequences comprising a sequence according to SEQ ID NO:1 or nucleic acid sequences derived from the amino acid sequences according to SEQ ID NO.: 2 to 10.

All of the nucleic acid sequences mentioned herein (single- and double-strand DNA and RNA sequences such as, for example, cDNA and mRNA) can be prepared from the nucleotide building blocks in a manner known per se by chemical synthesis, such as, for example, by fragment condensation of individual overlapping, complementary nucleic acid building blocks of the double helix. Oligonucleotides may be chemically synthesized, for example, in a known manner, by the phosphoramidite method (Voet, Voet, 2nd Edition, Wiley Press New York, pages 896-897). Annealing synthetic oligonucleotides and filling in gaps with the aid of the Klenow fragment of DNA polymerase, and ligation reactions and also general cloning methods are described in Sambrook et al. (1989), Molecular Cloning: A laboratory manual, Cold Spring Harbor Laboratory Press.

The invention also relates to nucleic acid sequences (single- and double-stranded DNA and RNA sequences such as cDNA and mRNA, for example) encoding any of the above polypeptides and their functional equivalents which are obtainable, for example, by using artificial nucleotide analogs.

The invention relates to both isolated nucleic acid molecules coding for polypeptides or proteins of the invention or for biologically active segments thereof and nucleic acid fragments which may be used, for example, for use as hybridization probes or primers for identifying or amplifying coding nucleic acids of the invention.

The nucleic acid molecules of the invention may moreover contain untranslated sequences of the 3′- and/or 5′-end of the coding region of the gene.

The invention furthermore comprises the nucleic acid molecules complementary to the specifically described nucleotide sequences or a section of said nucleic acid molecules.

The nucleotide sequences of the invention make possible the generation of probes and primers which can be used for identifying and/or cloning homologous sequences in other cell types and organisms. Such probes and primers usually comprise a nucleotide sequence region which hybridizes under “stringent” conditions (see hereinbelow) to at least about 12, preferably at least about 25, such as, for example, about 40, 50 or 75, consecutive nucleotides of a sense strand of a nucleic acid sequence of the invention or of a corresponding antisense strand.

An “isolated” nucleic acid molecule is removed from other nucleic acid molecules present in the natural source of the nucleic acid and may, in addition, be essentially free of other cellular materials or culture medium, if produced by recombinant techniques, or free of chemical precursors or other chemicals, if chemically synthesized.

A nucleic acid molecule of the invention may be isolated by means of standard techniques of molecular biology and the sequence information provided according to the invention. For example, cDNA can be isolated from a suitable cDNA library by using any of the specifically disclosed complete sequences or a section thereof as hybridization probe and standard hybridization techniques (as described, for example, in Sambrook, J., Fritsch, E. F. and Maniatis, T. Molecular Cloning: A Laboratory Manual. 2nd Ed., Cold Spring Harbor Laboratory, Cold Spring Harbor Laboratory Press, Cold Spring Harbor, N.Y., 1989). In addition, a nucleic acid molecule comprising any of the disclosed sequences or a section thereof can be isolated by polymerase chain reaction using the oligonucleotide primers generated on the basis of said sequence. The nucleic acid amplified in this way may be cloned into a suitable vector and characterized by DNA sequence analysis. The oligonucleotides of the invention may furthermore be prepared by standard methods of synthesis, for example using a DNA synthesizer.

The nucleic acid sequences of the invention can be identified and isolated in principle from all organisms. Advantageously, the nucleic acid sequences of the invention, such as SEQ ID NO: 1 or the homologs thereof, can be isolated from fungi, yeasts or bacteria. Bacteria which may be mentioned here are Gram-negative and Gram-positive bacteria. Preference is given to isolating the nucleic acids of the invention from Gram-negative bacteria, advantageously from alpha-proteobacteria, beta-proteobacteria or gamma-proteobacteria, particularly preferably from bacteria of the families Enterobacteriaceae, Pseudomonadaceae and Rhizobiaceae, very particularly preferably from bacteria of the generus Agrobacterium, Pseudomonas or Burkholderia, via methods known to the skilled worker.

Nucleic acid sequences of the invention, such as SEQ ID No: 1 or derivatives thereof, homologs or parts of the sequences, can be isolated from other fungi or bacteria, for example via genomic or cDNA libraries, by using, for example, common hybridization methods or the PCR technique. These DNA sequences hybridize with the sequences of the invention under standard conditions. For hybridization, it is advantageous to use short oligonucleotides of the conserved regions, for example of the active site, which can be identified via comparisons with L-carnitine dehydrogenase in a manner known to the skilled worker. However, it is also possible to use longer fragments of the nucleic acids of the present invention or the complete sequences for the hybridization. Said standard conditions vary depending on the nucleic acid used (oligonucleotide, longer fragment or complete sequence) or depending on the type of nucleic acids, DNA or RNA, being used for hybridization. Thus, for example, the melting temperatures of DNA:DNA hybrids are approx. 10° C. lower than those of DNA:RNA hybrids of the same length.

Standard conditions mean, for example, depending on the nucleic acid, temperatures between 42 and 58° C. in an aqueous buffer solution at a concentration of between 0.1 and 5×SSC (1×SSC=0.15 M NaCl, 15 mM sodium citrate, pH 7.2) or additionally in the presence of 50% formamide, for example 42° C. in 5×SSC, 50% formamide, The hybridization conditions for DNA:DNA hybrids are advantageously 0.1×SSC and temperatures between about 20° C. and 45° C., preferably between about 30° C. and 45° C. The hybridization conditions for DNA:RNA hybrids are advantageously 0.1×SSC and temperatures between about 30° C. and 55° C., preferably between about 45° C. and 55° C. These hybridization temperatures indicated are melting temperatures calculated by way of example for a nucleic acid of approx. 100 nucleotides in length and having a G+C content of 50% in the absence of formamide. The experimental conditions for DNA hybridization are described in relevant genetics textbooks, such as, for example, Sambrook et al., “Molecular Cloning”, Cold Spring Harbor Laboratory, 1989, and can be calculated using formulae known to the skilled worker, for example as a function of the length of the nucleic acids, the type of hybrid or the G+C content. Further information on hybridization can be found by the skilled worker in the following textbooks: Ausubel et al. (eds), 1985, Current Protocols in Molecular Biology, John Wiley & Sons, New York; Hames and Higgins (eds), 1985, Nucleic Acids Hybridization: A Practical Approach, IRL Press at Oxford University Press, Oxford; Brown (ed), 1991, Essential Molecular Biology: A Practical Approach, IRL Press at Oxford University Press, Oxford.

The invention also relates to derivatives of the specifically disclosed or derivable nucleic acid sequences.

Thus it is possible for further nucleic acid sequences of the invention to be derived, for example, from SEQ ID NO:1 and to differ therefrom by addition, substitution, insertion or deletion of one or more nucleotides, while still coding for polypeptides with the desired property profile.

The invention also comprises those nucleic acid sequences which comprise “silent mutations” or which are modified, in comparison with a specifically mentioned sequence, according to the codon usage of a specific source organism or host organism, and also naturally occurring variants thereof, such as splice variants or allelic variants, for example.

The invention also relates to sequences obtainable by conservative nucleotide substitutions (i.e. the amino acid in question is replaced with an amino acid of the same charge, size, polarity and/or solubility).

The invention also relates to the molecules derived from the specifically disclosed nucleic acids by way of sequence polymorphisms. These genetic polymorphisms may exist between individuals within a population, owing to natural variations. These natural variations usually cause a variance of from 1 to 5% in the nucleotide sequence of a gene.

Derivatives of the inventive nucleic acid sequence having the sequence SEQ ID NO: 1 mean, for example, allelic variants having at least 40% homology at the deduced amino acid level, preferably at least 60% homology, very particularly preferably at least 80% homology, over the entire sequence region (with regard to homology at the amino acid level, reference may be made to the above comments regarding polypeptides). Advantageously, the homologies may be higher across parts of the sequence.

Furthermore, derivatives also mean homologs of the nucleic acid sequences of the invention, in particular of SEQ ID NO: 1, for example fungal or bacterial homologs, truncated sequences, single-stranded DNA or RNA of the coding or noncoding DNA sequence. Thus, for example, homologs of SEQ ID NO: 1 at the DNA level are at least 40%, preferably at least 60%, particularly preferably at least 70%, very particularly preferably at least 80%, homologous over the entire DNA region indicated in SEQ ID NO: 1.

Moreover, derivatives mean, for example, fusions with promoters. Said promoters which are located upstream of the nucleotide sequences may have been modified by one or more nucleotide substitutions, insertions, inversions and/or deletions, without adversely affecting the functionality or efficacy of the said promoters, however. Furthermore, the efficacy of said promoters may be increased by modifying their sequence, or said promoters may be replaced completely with more efficient promoters, including those of organisms of different species.

Derivatives also mean variants whose nucleotide sequence in the region of from −1 to −1000 bases upstream of the start codon or from 0 to 1000 bases downstream of the stop codon has been altered so as to modify, preferably increase, gene expression and/or protein expression.

Furthermore, the invention also comprises nucleic acid sequences which hybridize with the abovementioned coding sequences under “stringent conditions”. These polynucleotides can be identified when screening genomic or cDNA libraries and, where appropriate, be amplified therefrom by means of PCR using suitable primers and subsequently isolated using suitable probes, for example. In addition, polynucleotides of the invention may also be synthesized chemically. This property means the ability of a poly- or oligonucleotide to bind under stringent conditions to a virtually complementary sequence, while there are no nonspecific bindings between noncomplementary partners under these conditions. For this purpose, the sequences should be 70-100%, preferably 90-100%, complementary. The property of complementary sequences of being able to bind specifically to one another is exploited, for example, in the Northern or Southern blot technique or for primer binding in PCR or RT-PCR. For this purpose, oligonucleotides from a length of 30 base pairs are customarily used. Stringent conditions mean, for example in the Northern blot technique, using a washing solution, for example 0.1×SSC buffer containing 0.1% SDS (20×SSC: 3M NaCl, 0.3M sodium citrate, pH 7.0) at a temperature of 50-70° C., preferably 60-65° C., for eluting nonspecifically hybridized cDNA probes or oligonucleotides. In the process, only highly complementary nucleic acids remain bound to one another, as mentioned above. Setting of stringent conditions is known to the skilled worker and described, for example, in Ausubel et al., Current Protocols in Molecular Biology, John Wiley & Sons, N.Y. (1989), 6.3.1-6.3.6.

E. Constructs of the Invention

The invention moreover relates to expression constructs comprising, under the genetic control of regulatory nucleic acid sequences, a nucleic acid sequence coding for a polypeptide of the invention; and also to vectors comprising at least one of said expression constructs.

Such constructs of the invention preferably comprise a promoter 5′ upstream of the particular coding sequence and a terminator sequence 3′ downstream and also, where appropriate, further common regulatory elements, in each case operatively linked to the coding sequence.

An “operative linkage” means the sequential arrangement of promoter, coding sequence, terminator and, where appropriate, further regulatory elements in such a way that each of said regulatory elements is able to carry out its function in expression of the coding sequence. Examples of operatively linkable sequences are targeting sequences and also enhancers, polyadenylation signals, and the like. Other regulatory elements comprise selectable markers, amplification signals, origins of replications, and the like. Suitable regulatory sequences are described, for example, in Goeddel, Gene Expression Technology: Methods in Enzymology 185, Academic Press, San Diego, Calif. (1990).

A nucleic acid construct of the invention means in particular the L-carnitine dehydrogenase genes having the sequence SEQ ID NO: 1 and the derivatives and homologs thereof and also the nucleic acid sequences derivable from SEQ ID NO: 2 to 10, which advantageously have been operatively or functionally linked to one or more regulatory signals for controlling, for example increasing, gene expression.

In addition to these regulatory sequences, the natural regulation of said sequences may still be present upstream of the actual structural genes and, where appropriate, may have been genetically modified so that natural regulation has been switched off and expression of the genes has been increased. However, construction of the nucleic acid construct may also be simpler, i.e. no additional regulatory signals have been inserted upstream of the coding sequence (such as, for example, SEQ ID NO: 1 or its homologs), and the natural promoter together with its regulation has not been removed. Instead, the natural regulatory sequence has been mutated in such as way that regulations no longer takes place and gene expression is increased.

A preferred nucleic acid construct advantageously also comprises one or more of the already mentioned enhancer sequences, functionally linked to the promoter, which enable expression of the nucleic acid sequence to be increased. Additional advantageous sequences such as further regulatory elements or terminators may also be inserted at the 3′ end of the DNA sequences. One or more copies of the nucleic acids of the invention may be present in the construct. The construct may also contain other markers such as resistances to antibiotics or auxotrophy-complementing genes, where appropriate, for selection for the construct.

Advantageous regulatory sequences for the method of the invention are present, for example, in promoters such as cos, tac, trp, tet, trp-tet, lpp, lac, lpp-lac, lacl^{q,} T7, T5, T3, gal, trc, ara, rhaP (rhaP_{BAD})SP6, lambda-P_{R} or in the lambda-P_{L} promoter, which are advantageously used in Gram-negative bacteria. Other advantageous regulatory sequences are present, for example, in the Gram-positive promoters amy and SPO2, in the yeast or fungal promoters ADC1, MFalpha , AC, P-60, CYC1, GAPDH, TEF, rp28, ADH. In this connection, the promoters of pyruvate decarboxylase and of methanol oxidase, for example from Hansenula, are also advantageous. It is also possible to use artificial promoters for regulation.

The nucleic acid construct is advantageously expressed in a host organism by inserting it into a vector such as a plasmid or a phage, for example, which makes possible optimal expression of the genes in the host. Vectors mean, apart from plasmids and phages, also any other vectors known to the skilled worker, i.e., for example, viruses such as SV40, CMV, Baculovirus and Adenovirus, Transposons, IS elements, phasmids, cosmids, and linear or circular DNA. These vectors can be replicated autonomously in the host organism or chromosomally. These vectors constitute a further embodiment of the invention. Suitable plasmids are, for example, in E. coli pLG338, pACYC184, pBR322, pUC18, pUC19, pKC30, pRep4, pHS1, pKK223-3, pDHE19.2, pHS2, pPLc236, pMBL24, pLG200, pUR290, pIN-III¹¹³-B1, λgt11 or pBdCl, in Streptomyces pIJ101, pIJ364, pIJ702 or pIJ361, in Bacillus pUB110, pC194 or pBD214, in Corynebacterium pSA77 or pAJ667, in fungi pALS1, pIL2 or pBB116, in yeasts 2alphaM, pAG-1, YEp6, YEp13 or pEMBLYe23 or in plants pLGV23, pGHlac⁺, pBIN19, pAK2004 or pDH51. The plasmids mentioned are a small selection of possible plasmids. Other plasmids are well known to the skilled worker and can be found, for example, in the book Cloning Vectors (Eds. Pouwels P. H. et al. Elsevier, Amsterdam-New York-Oxford, 1985, ISBN 0 444 904018).

For expression of the further genes which are present, the nucleic acid construct advantageously additionally comprises 3′- and/or 5′-terminal regulatory sequences for enhancing expression, which are selected for optimal expression depending on the host organism and the gene or genes selected.

These regulatory sequences are intended to make possible specific expression of the genes and protein expression. Depending on the host organism, this may mean, for example, that the gene is expressed or overexpressed only after induction or that it is immediately expressed and/or overexpressed.

The regulatory sequences or factors may preferably have a positive effect on, and thus increase, gene expression of the genes introduced. Thus, the regulatory elements can advantageously be enhanced at the transcriptional level by using strong transcription signals such as promoters and/or enhancers. However, in addition it is also possible to enhance translation by improving, for example, stability of the mRNA.

In a further embodiment of the vector, the vector comprising the nucleic acid construct of the invention or the nucleic acid of the invention may advantageously also be introduced into the microorganisms in the form of a linear DNA and integrated via heterologous or homologous recombination into the genome of the host organism. This linear DNA may consist of a linearized vector such as a plasmid or only of the nucleic acid construct or the nucleic acid of the invention.

It is advantageous for optimal expression of heterologous genes in organisms to modify the nucleic acid sequences according to the specific codon usage used in the organism. The codon usage can be readily determined on the basis of computer evaluations of other, known genes of the organism in question.

The expression cassette of the invention is prepared by fusing a suitable promoter to a suitable coding nucleotide sequence and a terminator signal or polyadenylation signal. For this purpose, conventional recombination and cloning techniques are used, as are described, for example, in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley Interscience (1987).

The recombinant nucleic acid construct or gene construct is expressed in a suitable host organism by inserting it advantageously into a host-specific vector which makes optimal expression of the genes in the host possible. Vectors are well known to the skilled worker and can be found, for example, in “Cloning Vectors” (Pouwels P. H. et al., eds., Elsevier, Amsterdam-New York-Oxford, 1985).

F. Hosts which are Usable According to the Invention

It is possible with the aid of the vectors of the invention to prepare recombinant microorganisms which are transformed, for example, with at least one vector of the invention and can be used for producing the polypeptides of the invention. The above-described recombinant constructs of the invention are advantageously introduced into a suitable host system and expressed. In this context, preference is given to using cloning and transfection methods familiar to the skilled worker, such as, for example, coprecipitation, protoplast fusion, electroporation, retroviral transfection and the like, in order to express the nucleic acids mentioned in the particular expression system. Suitable systems are described, for example, in Current Protocols in Molecular Biology, F. Ausubel et al., Eds., Wiley Interscience, New York 1997, or Sambrook et al. Molecular Cloning: A Laboratory Manual. 2nd Ed., Cold Spring Harbor Laboratory, Cold Spring Harbor Laboratory Press, Cold Spring Harbor, N.Y., 1989.

According to the invention, it is also possible to prepare microorganisms by homologous recombination. For this purpose, a vector is prepared which contains at least one section of a gene of the invention or of a coding sequence, into which at least one amino acid deletion, addition or substitution has been introduced, where appropriate, in order to modify, for example to functionally disrupt, the sequence of the invention (“knockout” vector). The sequence introduced may also be, for example, a homolog from a related microorganism or may have been derived from a mammalian, yeast or insect source. Alternatively, the vector used for homologous recombination may be designed in such a way that the endogenous gene is mutated or otherwise modified upon homologous recombination, while still encoding the functional protein (for example, the upstream regulatory region may have been modified in such a way that this causes modified expression of the endogenous protein). The modified section of the gene of the invention is present in the homologous recombination vector. The construction of suitable vectors for homologous recombination is described, for example, in Thomas, K. R. and Capecchi, M. R. (1987) Cell 51:503.

Suitable recombinant host organisms for the nucleic acid of the invention or the nucleic acid construct are, in principle, all prokaryotic or eukaryotic organisms. Host organisms which are used advantageously are microorganisms such as bacteria, fungi or yeasts. Advantageously used are Gram-positive or Gram-negative bacteria, preferably bacteria of the families Enterobacteriaceae, Pseudomonadaceae, Rhizobiaceae, Streptomycetaceae and Nocardiaceae, particularly preferably bacteria of the genera Escherichia, Pseudomonas, Streptomyces, Nocardia, Burkholderia, Salmonella, Agrobacterium and Rhodococcus. Very particular preference is given to the genus and species Escherichia coli. In addition, further advantageous bacteria can be found in the group of alpha-proteobacteria, beta-proteobacteria or gamma-proteobacteria.

The host organism, or host organisms, according to the invention comprise at least one of the nucleic acid sequences, nucleic acid constructs or vectors which are described in the present invention and which code for an enzyme having L-carnitine dehydrogenase activity (Kleber H P (1997) FEMS Microbiology, 147, 1-9).

Depending on the host organism, the organisms used in the method of the invention are cultured or grown in a manner known to the skilled worker. Microorganisms are usually grown in a liquid medium comprising a carbon source, usually in the form of sugars, a nitrogen source, usually in the form or organic nitrogen sources such as yeast extract or of salts such as ammonium sulfate, trace elements such as salts of iron, manganese, magnesium, and, where appropriate, vitamins, at temperatures between 0° C. and 100° C., preferably between 10° C. and 60° C., while passing in oxygen. The pH of the nutrient liquid may be kept constant there, i.e. regulated or not regulated during cultivation. Cultivation may be batchwise, semibatchwise or continuous. Nutrients may be introduced at the start of the fermentation or fed in semicontinuously or continuously. The ketone may be added directly to the cultivation or, advantageously, after cultivation. The enzymes may be isolated from the organisms by the method described in the examples or used as crude extract for the reaction.

The host organisms contain advantageously 1 U/I enzyme activity, for example L-carnitine dehydrogenase activity, preferably 100 U/I, particularly preferably more than 1000 U/I.

G. Recombinant Production of the Polypeptides

The invention furthermore relates to methods for recombinant production of polypeptides of the invention or of functional, biologically active fragments thereof, in which method a polypeptide-producing microorganism is cultured, expression of said polypeptides is induced where appropriate, and the latter are isolated from the culture. If desired, the polypeptides may also be produced on the industrial scale in this manner.

The recombinant microorganism can be cultured and fermented by known methods. For example, bacteria may be propagated in TB or LB medium and at a temperature of from 20 to 40° C. and pH 6 to 9. Suitable culturing conditions are described in detail, for example, in T. Maniatis, E. F. Fritsch and J. Sambrook, Molecular Cloning: A Laboratory Manual, Cold Spring Harbor Laboratory, Cold Spring Harbor, N.Y. (1989).

Unless the polypeptides are secreted into the culture medium, the cells are then disrupted and the product is obtained from the lysate by known protein isolation methods. The cells can be disrupted either by high-frequency ultrasound, by high pressure, for example in a French press, by osmolysis, by the action of detergents, lytic enzymes or organic solvents, by homogenizers or by combining two or more of the methods listed.

The polypeptides can be purified using known chromatographic methods such as molecular sieve chromatography (gel filtration), for example Q-Sepharose chromatography, ion exchange chromatography and hydrophobic chromatography, and also by other customary methods such as ultrafiltration, crystallization, salting out, dialysis and native gel electrophoresis. Suitable methods are described, for example, in Cooper, T. G., Biochemische Arbeitsmethoden [The Tools of Biochemistry], Verlag Walter de Gruyter, Berlin, New York or in Scopes, R., Protein Purification, Springer Verlag, New York, Heidelberg, Berlin.

It may be advantageous to isolate the recombination protein by using vector systems or oligonucleotides which extend the cDNA by particular nucleotide sequences and thus code for modified polypeptides or fusion proteins which simplify purification, for example. Suitable modifications of this kind are, for example, “tags” acting as anchors, such as, for example, the modification known as hexa-histidine anchor, or epitopes which can be recognized as antigens by antibodies (described, for example, in Harlow, E. and Lane, D., 1988, Antibodies: A Laboratory Manual. Cold Spring Harbor (N.Y.) Press). These anchors can be used for attaching the proteins to a solid support such as, for example, a polymer matrix which may be packed, for example, in a chromatography column or on a microtiter plate or on any other support.

At the same time, these anchors may also be used for identifying the proteins. Moreover, customary labels such as fluorescent dyes, enzyme labels which, after reaction with a substrate, form a detectable reaction product or radiolabels may be used, alone or in combination with said anchors, for identifying the proteins in order to derivatize said proteins.

H. Carrying out the Method of the Invention for Preparing (S)-alkanols

The enzymes having L-carnitine dehydrogenase activity, such as carnitine dehydrogenase or hydroxyacyl-CoA dehydrogenase, may be used as free or immobilized enzyme in the method of the invention.

The method of the invention is advantageously carried out at a temperature between 0° C. and 95° C., preferably between 10° C. and 85° C., particularly preferably between 15° C. and 75° C.

The pH in the method of the invention is advantageously maintained between pH 4 and 12, preferably between pH 4.5 and 9, particularly preferably between pH 5 and 8.

In the method of the invention, enantiomerically pure or chiral products such as 3-methylamino-1-(2-thienyl)-(S)-propanol mean enantiomers which show enrichment of one enantiomer. The method preferably achieves enantiomeric purities of at least 70% ee, preferably of at least 80% ee, particularly preferably of at least 90% ee, very particularly preferably of at least 98% ee.

It is possible to use for the method of the invention growing cells which comprise the nucleic acids, nucleic acid constructs or vectors of the invention. It is also possible to use resting or disrupted cells. Disrupted cells mean, for example, cells which have been made permeable by treatment with, for example, solvents, or cells which have been ruptured by an enzyme treatment, by a mechanical treatment (for example French Press or ultrasound) or by another method. The crude extracts obtained in this way are advantageously suitable for the method of the invention. Purified or partially purified enzymes may also be used for the method. Likewise suitable are immobilized microorganisms or enzymes which can advantageously be utilized in the reaction.

If free organisms or enzymes are used for the method of the invention, these are expediently removed, for example by filtration or centrifugation, before the extraction.

The product prepared in the method of the invention, such as 3-methylamino-1-(2-thienyl)-(S)-propanol, can advantageously be isolated from the aqueous reaction solution by extraction or distillation or, advantageously, by extraction and distillation. The extraction can be repeated several times to increase the yield. Examples of suitable extractants are solvents such as toluene, methylene chloride, butyl acetate, diisopropyl ether, benzene, MTBE or ethyl acetate, without being limited thereto.

After concentration of the organic phase, the products can usually be obtained in good chemical purities, i.e. greater than 80% chemical purity. After extraction, the organic phase containing the product can, however, also be only partly concentrated, and the product can be crystallized out. For this purpose, the solution is advantageously cooled to a temperature of from 0° C. to 10° C. Crystallization is also possible directly from the organic solution or from an aqueous solution. The crystallized product can be taken up again in the same or in a different solvent for recrystallization and be crystallized again. It is possible, by carrying out the subsequent advantageous crystallization at least once, to increase the enantiomeric purity of the product further if necessary.

With the types of workup mentioned, the product of the method of the invention can be isolated in yields of from 60 to 100%, preferably from 80 to 100%, particularly preferably from 90 to 100%, based on the substrate employed for the reaction, such as 3-methylamino-1-(2-thienyl)-propan-1-one, for example. The isolated product is distinguished by a high chemical purity of >90%, preferably >95%, particularly preferably >98%. Furthermore, the products have a high enantiomeric purity which can advantageously be further increased, if necessary, by said crystallization.

The method of the invention can be carried out batchwise, semibatchwise or continuously.

The method may advantageously be carried out in bioreactors as described, for example, in Biotechnology, Volume 3, 2nd Edition, Rehm et al. Eds., (1993), in particular Chapter II.

The description above and the examples below serve only to illustrate the invention. The invention likewise comprises the numerous possible modifications obvious to the skilled worker.

### EXPERIMENTAL SECTION

### Example 1

### Cloning of Carnitine Dehydrogenases or Hydroxyacyl-CoA Dehydrogenases via PCR Amplification

Bacteria selected from the genera Alcaligenes, Pseudomonas, Xanthomonas, Agrobacterium, Mesorhizobium and Rhizobium, Streptomyces and Archaeglobus were cultivated in 25 ml of complex medium (e.g. HFP=1% peptone, 1% tryptone, 0.5% yeast extract, 0.3% NaCl) for 1-3 days, harvested, washed in buffer, resuspended (5 ml of 50 mM Tris pH 7.0), and the genomic DNA was prepared with the aid of the QIAGEN genomic tip system from Qiagen. The carnitine dehydrogenase and 3-hydroxyacyl-CoA dehydrogenase genes were then amplified by means of PCR. To this end, the DNA sequences available to the skilled worker, belonging to the dehydrogenase sequences of SEQ ID 2-10, were selected from the N- and C-terminus (in each 25-30 bp), restriction cleavage sites for cloning were optionally attached thereto, and the corresponding oligonucleotides were synthesized. The PCR reaction was carried out using Pfu polymerase (Stratagene) or Taq polymerase (Roche). For example, the following temperature program was carried out for the PCR reactions:

95° C. for 3 minutes; 30 cycles with 95° C. for 45 s, 55° C. for 45 s and 72° C. for 2 min 50 s; 72° C. for 10 min; storage at 4° C. until first use. All PCR products were purified by agarose gel electrophoresis (E-Gel, Invitrogen) and column chromatography (GFX-Kit, Pharmacia). Cloning into vectors such as pBluescript KSII, pKK223-3 and pDHE19.2 was carried out using suitable restriction digests and ligation according to Maniatis T, Fritsch E F and Sambrook J (1989) Molecular Cloning: A Laboratory Manual, Cold Spring Harbor Laboratory, Cold Spring Harbor (N.Y.). Taq-PCR products were cloned into the pBADtopo vector directly. Suitable host organisms used were E.coli XL1 B and TG1 (Stratagene).

### Example 2

### Cloning of the Carnitine Dehydrogenases or Hydroxyacyl-CoA Dehydrogenases by Growth Selection

Organisms of the genera mentioned in Example 1 and other bacteria, yeasts and fungi and also isolates from soil samples and E.coli gene libraries prepared by cloning DNA from soil samples were stripped out on suitable minimal media containing carnitine or methylamino-1-(2-thienyl)-(S)-propanol, for example 1% D,L-carnitine, 0.2% K₂HPO₄, 0.05% MgSO₄·7 H₂O, 0.05% yeast extract, or incubated in liquid medium, and, after one day, three days, once a week or after one month, (repeatedly) transferred to fresh medium by inoculation. The organisms multiplied in this way were isolated in the form of single colonies or by sorting in a cell sorter. They showed the ability to grow on carnitine or methylamino-1-(2-thienyl)-(S)-propanol as sole carbon and/or nitrogen source. It was possible to use the strains obtained to generate new recombinant dehydrogenase strains via PCR amplification (according to Example 1).

### Example 3

### Conversion of Methylamino-1-(2-thienyl)-(S)-propanol

Biomass of the strains obtained in Examples 1 and 2 was harvested after cultivation in the presence of suitable inducers (e.g. 0.5 mM IPTG, 2 g/L rhamnose, carnitine), washed in buffer (e.g. 50 mM Tris-HCl pH 7.0) and resuspended, and the resting cells were admixed with NADH or NADPH (0.1-5 mM), 1.6 mg-50 mg of methylamino-1-(2-thienyl)-(S)-propanol and either glucose or isopropanol (1-100 mol eq.) per ml of reaction mixture and incubated at 30° C. for 1-24 h. The reaction could be monitored by way of decrease in extinction at 340 nm or by HPLC analysis. The strains had activities of between 0 and 100 U/I.

1. A method for the microbiological preparation of substituted (S)-alkanol compounds of formula I 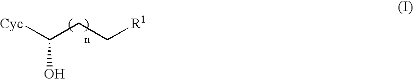
in which
n is an integer from 0 to 5;
Cyc is an unsubstituted or substituted, mono- or polynuclear, saturated or unsaturated, carbocyclic or heterocyclic ring, and
R¹ is halogen, SH, OH, NO₂, NR²R³ or NR²R³R⁴⁺X⁻, where R², R³ and R⁴ independently of one another are H or a lower alkyl or lower alkoxy radical and X⁻ is a counterion, which comprises
a) culturing a microorganism which produces an enzyme having L-carnitine dehydrogenase activity, or
b) incubating an enzyme having L-carnitine dehydrogenase activity, in a medium containing an alkanone compound of formula II 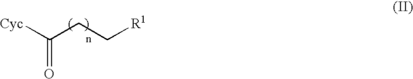
in which n, Cyc and R¹ are as defined above, such that
the compound of formula II is enzymatically reduced to give the compound of formula I, and
c) isolating the product formed.

2. The method of claim 1, for preparing a compound of formula I, wherein Cyc is an optionally substituted mononuclear, saturated or unsaturated carboxyclic or heterocyclic ring.

3. The method as claimed in claim 1, wherein the substituted (S)-alkanol compound is 3-methylamino-1-(2-thienyl)-(S)-propanol of formula III 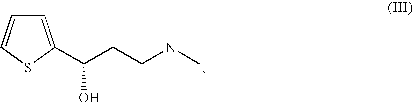
the alkanone compound in the medium is 3-methylamino-1-(2-thienyl)-propan-2-one of formula IV 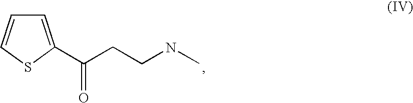
the compound of formula IV is enzymatically reduced to give the compound of formula III, and the product formed is essentially enantiomerically pure.

4. The method as claimed in claim 1, wherein the enzyme having L-carnitine dehydrogenase activity is selected from among L-carnitine dehydrogenases (E.C. 1.1.1.108) and 3-hydroxyacyl-CoA dehydrogenases (E.C. 1.1.1.35).

5. The method as claimed in claim 1, wherein the enzyme having L-carnitine dehydrogenase activity is selected from among enzymes of microorganisms of the genera Alcaligenes, Pseudomonas, Xanthomonas, Staphylococcus, Rhizobium, Agrobacterium, Streptomyces and Archaeglobus.

6. The method as claimed in claim 1, wherein the enzyme having L-carnitine dehydrogenase activity is selected from among enzymes comprising an amino acid sequence according to SEQ ID NO: 2, 3, 4, 5, 6, 7, 8, 9 or 10 or encoded by nucleic acid sequences derived therefrom; and functional equivalents of said enzymes, which have L-carnitine dehydrogenase activity and catalyze the enantioselective synthesis of a compound of formula I.

7. The method as claimed in claim 1, wherein the enzyme having L-carnitine dehydrogenase activity is encoded by a nucleic acid sequence according to SEQ ID NO: 1 or a functional equivalent thereof.

8. The method as claimed in claim 1, wherein the enzymatic reduction is carried out with addition of reduction equivalents or under conditions in which the reduction equivalents consumed are regenerated.

9. The method as claimed claim 1, wherein the compound of formula II is reacted in the presence of a microorganism selected from among the bacteria of the families Enterobacteriaceae, Pseudomonadaceae, Rhizobiaceae, Streptomycetaceae and Nocardiaceae.

10. The method as claimed in claim 1, wherein the microorganism is a recombinant microorganism which has been transformed with a nucleic acid construct coding for an enzyme having L-carnitine dehydrogenase activity.

11. The method as claimed in claim 1, wherein
a) the microorganism producing an enzyme having L-carnitine dehydrogenase activity is isolated from a natural source or is prepared recombinantly,
b) said microorganism is propagated,
c) said enzyme having L-carnitine dehydrogenase activity is, where appropriate, isolated from said microorganism or a protein fraction containing said enzyme is prepared from said microorganism, and
d) said microorganism according to stage b) or said enzyme according to stage c) is transferred into a medium containing a compound of formula I.

12. A method for preparing a compound of formula V 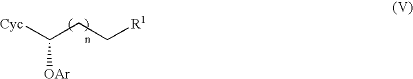
in which
n is an integer from 0 to 5;
Cyc is an unsubstituted or substituted, mono- or polynuclear, saturated or unsaturated, carbocyclic or heterocyclic ring, and
R¹ is halogen SH, OH, NO₂, NR²R³ or NR²R³R⁴⁺X⁻, where R², R³ and R⁴ independently of one another are H or a lower alkyl or lower alkoxy radical and X⁻ is a counterion,
and Ar is a mono- or polynuclear, unsubstituted or substituted aromatic radical, comprising
a) a preparing microbiologically the compound of formula I as defined in claim 1; and
b) reacting the compound of formula I with an aromatic compound of formula VI
Ar—Y (VI)
in which Ar is as defined above and Y is a leaving group, and
c) isolating the compound of formula V.

13. The method as claimed in claim 12, wherein Ar of the compound of formula V in is 1-naphthyl, Cyc is 2-thienyl, R¹ is monomethylamino and n is 1.

14. A polypeptide which comprises an amino acid sequence according to SEQ ID NO: 2, 3, 4, 5, 6, 7, 8, 9 or 10 or is encoded by nucleic acid sequences derived therefrom; or of a functional equivalent of these enzymes, which have L-carnitine dehydrogenase activity and catalyze the enantioselective synthesis of a compound of formula I, for the microbial or enzymatic synthesis of substituted (S)-alkanols of formula I.

15. A nucleic acid sequence comprising the sequence coding for a polypeptide as claimed in claim 14.

16. (canceled)

17. The method as claimed in claim 1 wherein the compound prepared is Duloxetine.

18. An expression cassette comprising the nucleic acid sequence as claimed in claim 15, which sequence is operatively linked to at least one regulatory nucleic acid sequence.

19. A recombinant vector comprising at least one expression cassette as claimed in claim 18.

20. A prokaryotic or eukaryotic host transformed with at least one vector as claimed in claim 19.


SEQUENCE LISTING (10 sequences)

SEQ ID NO: 1 - DNA, 966 bp, Alcaligenes sp.
  CDS (1)..(966)
atgacttttatcaccaacatcaaaaccttcgccgctctgggcagcggcgtgatcggcagtggctgggtggcccgcgccctggcccacggcctggacgtggtggcctgggacccggcgccgggcgctgaagccgccctgcgccagcgggtcgccaacgcctggggcgctctggaaaaacagggcctggtccccggagcctcgccacagcgcctacgctttgtcagcaccatcgaagagtgtgtgaaagacgccgacttcatccaggaaagcgctcccgagcgcctggaactcaagctggaactgcacagcaagatcagcgccgcggccaagcccgacgccctgatcggctccagcacctcggccctgctgcccagcgaattctatgagggctcgacccacccgcaacgctgcgtggtgggccatccgttcaacccggtgtacctgctaccgctggtggaagtggtgggcggccagcacaccgcacccgaggcgatacaggcggcgatccaggtctatgaatccttggggatgcgccccttgcatgtgcgcaaggaagtcccgggcttcatcgccgaccgtctgctcgaagccctgtggcgcgaagccttgcacctggtcaatgacggggtggcgaccaccggggaaatcgacgacgcgatccgctttggcgccggcctgcgctggtcgttcatgggcaccttcctcacctacaccctggccggaggcgacgccggcatgcgtcacttcatggcccagttcggcccggcgctgcaactgccctggacctacctgccggccccggagctgaccgacaaactgatcgatgacgtagtccaaggtaccgccgagcaactgggcaaccacagcatttcggccctggagcgctatcgtgatgactgcctgctggcggtgctggaggcggtgaaaaccaccaaggccaagcacggcatgcactttcacgactaa

SEQ ID NO: 2 - protein, 321 aa, Alcaligenes sp.
MTFITNIKTFAALGSGVIGSGWVARALAHGLDVVAWDPAPGAEAALRQRVANAWGALEKQGLVPGASPQRLRFVSTIEECVKDADFIQESAPERLELKLELHSKISAAAKPDALIGSSTSALLPSEFYEGSTHPQRCVVGHPFNPVYLLPLVEVVGGQHTAPEAIQAAIQVYESLGMRPLHVRKEVPGFIADRLLEALWREALHLVNDGVATTGEIDDAIRFGAGLRWSFMGTFLTYTLAGGDAGMRHFMAQFGPALQLPWTYLPAPELTDKLIDDVVQGTAEQLGNHSISALERYRDDCLLAVLEAVKTTKAKHGMHFHD

SEQ ID NO: 3 - protein, 321 aa, Pseudomonas putida
MPFITEIKTFAALGSGVIGSGWVARALAHGLDVVAWDPAPGAEQALRKRVANAWPALEKQGLAPGASQDRLKFVATIEECVRNADFIQESAPERLDLKLDLHAKISAAAKPDAIIGSSTSGLLPSEFYESSTHPERCVVGHPFNPVYLLPLVEIVGGSRTSPEAIEAAKTIYTALGMRPLHVRKEVPGFIADRLLEALWREALHLVNDGVATTGEIDDAIRFGAGLRWSFMGTFLTYTLAGGDAGMRHFMSQFGPALKLPWTYLPAPELTDKLIDDVVSGTSEQQGERSIAALERYRDDTLLAVLEAVKSSKASHGLSFSD

SEQ ID NO: 4 - protein, 321 aa, Pseudomonas aeruginosa
MSFVTEIKTFAALGSGVIGSGWIARALAHGLDVVAWDPAPGAEAALRARVANAWPALRKQGLAPGAAQERLRFVASIEECVGDADFIQESAPERLDLKLDLHARISAAARPDVLIGSSTSGLLPSEFYAEASHPERCLVGHPFNPVYLLPLVEVVGGERTAAEAVRAAMRVYESLGMRPLHVRKEVPGFIADRLLEALWREALHLVNDGVATTGEIDDAIRFGAGLRWSFMGTFLTYTLAGGNAGMRHFMAQFGPALQLPWTYLPAPELTEALIDRVVEGTAEQQGARSIAELERYRDDCLLAVLGAIRETKARHGFAFAE

SEQ ID NO: 5 - protein, 256 aa, Xanthomonas campestris
MQPSSVLAIVTGGVSGLGLAVAQRIVADGGRVALFDVNADKADAALALLGAGNAHYFATDVTDEAQVAANVAQAAQALGGLNLAVNCAGILGAGRVLGKEAAMPLATFRTTVMVNLVGSFNVAKAAADVMQHNAAGEDGERGVIIHTASVAAYEGQIGQAAYAASKGGVVGMTLPMARELARFGIRVMTIAPGVFWTPMLDGMPETVQQSLAASIPFPSRLGQPDEYAETVAFILRTRYLNGEVIRLDGGVRLAPK

SEQ ID NO: 6 - protein, 321 aa, Staphylococcus epidermidis
MKFAVVGTGVIGSGWITRMLAHGHEVIATDPSEGAYERMLTQVKQNWPYAEQMGLAENASIQNLTFTPHLEEAVKDADHIQENVPEVEEIKDAVLKEIDFYAKPEATIGSSTSGIMPSELQANLSHPERLVVAHPFHPVYILPLVEIVPGKQTSEETTVKAEQIYESIGMDVLHVRHEIEGHIADRLMEALWRESLHIVNDGIATTEEVDKAFTHAAGLRYAQYGPFMTFHLAGGEGGMRHMLKQFGPALKKPWTKLIAPELTDDLYHKVVSGSEASSQGYTMSELDQKRNEFLIKVKELAEQYWPSDSKAMKKSNGAELQ

SEQ ID NO: 7 - protein, 364 aa, Rhizobium loti
MSIINKAAAIGGGVIGAGWVARLLLNGIDVSIFDPDPEASRKVSEVMKGARRAYKQMVPGGLPKEGKLTFAKTIAEAVADADFIQESVPERLDLKHRVLAEIDAHAPANAIVGSSTSGIKPTDMQVAMKKHPERLVVGHPFNPVYLLPLVEIVGGDQTFPEAIEVAKEIYASIGMKPVVIRKEIEAFVGDRLLEAAWREALWLIKDGICTVEELDDIMRYGFGLRWAQMGMFQVYRVAGGEAGMRHFMAQFGPCLKWPWTKLMDVPEFNDELVDLIATQSDDQAHGLSIRELEKIRDDNLVAIMDALSKQNKGKGWGAGALHKDYTKQLAKLAAKKPAASTAAEKAKASKPVKKAEKPKKKKKG

SEQ ID NO: 8 - protein, 488 aa, Agrobacterium tumefaciens
MVAGMKIAAIGGGVIGGGWIARFILAGHDVTVFDPHPEANRIVTEVMANAAEAWGRLYQSPLPKPGSINWASSIAEAVAGADYIQESVPERLDLKHRIIAEIEATASPQAIIASSTSGFKPSELREGSVHSERVIVAHPFNPVYLLPVVEVVGGGVAAQRASDILVSVGMKPVQIGREIDAHIGDRLLEAIWREALWLVKDGIATTQEIDDIIRYGFGLRWAQMGLFETYRIAGGEAGMRHFLAQFGPALKWPWTKLMDVPEFNDELIDLIAGQSDAQSGAYSIRELERIRDSNLIGIFHALKANDWGAGQTVKAMENRFYARNGKVQADYPLRLHEAHVNGGWVDYNGHMTEFRYLQVLGDATDALLIHIGLDADYRAAGHSAYTVETHIRHLAEVKAGARLTVETRLLGYDDKRLRLHHAILNEDGETVATGEHMLLHVDTKANRTVAMPPALMRALDHLNAQEEGPLPDHAGSGIRAVRLKETSA

SEQ ID NO: 9 - protein, 307 aa, Streptomyces coelicolor
MSTVTVIGAGTIGLGWINLFSARGLTVRVNSRRPDVRRVVHEALELFSPGRVDELAARIEYEPDVGRAVAGADVVSENAPDDLPLKQRLFAEIGAAAPDHALVLSSTSKLLPDELSRDMPGPGRLVVAHPFNPPHIVPLVEVVRGERTDPEAVERTLAFLASVGRTPVVVRRALPGFAANRLQSALLRESIHLVLEGVVTVEELDRIVTDSIGLRWSTIGPFHAFHLGGGPGGLRKWLEHLGSGLEQGWRGLGQPALTPQAVEALVAQTEAAYGHRPYAELVRDRDDRHLAVLAALERTEQPQEETK

SEQ ID NO: 10 - protein, 315 aa, Archaeoglobus fulgidus
MGVKVACIGAGTVGASWASLFAWRGCDVAVYDPFPEALNRAEASIARTVSTLSEIFSGSEDDVKSALSRVKFTENLEEALKGAYYVQESAVEKLEVKRDLFEKMDAIAEPETILATSTSGLSISEIQTAARKHPERCITAHPYNPPHLIPLVEVVPRKQTDESCTEKTVEFMERMGKKPIVVKKDVPGMVANRLAAALWREAVNLVYQGIATPEEIDVAVKYGPGIRWAITGVYLTYHLGGGQGGMKYFLEFFDDHYRRLWSDLANWSEYPEGSFDAILKSMEGYDIIKRMSYDELAKWRDDQLAKILKLILEDG